(19) United States
(12) Patent Application Publication
(10) Pub. No.: US 2009/0299089 A1
(43) Pub. Date: Dec. 3, 2009

(54) METHOD FOR PRODUCING OPTICALLY ACTIVE 3-AMINOCARBOXYLIC ACID ESTERS

(75) Inventors: Christoph Jaekel, Limburgerhof (DE); Wolf-Ruediger Krahnert, Schifferstadt (DE); Rocco Paciello, Bad Duerkheim (DE); Wolfgang Siegel, Limburgerhof (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/307,472

(22) PCT Filed: Jul. 5, 2007
(86) PCT No.: PCT/EP2007/056843

§ 371 Date: Jan. 5, 2009

(30) Foreign Application Priority Data
Jul. 6, 2006 (EP) 06116717.7

(51) Int. Cl. C07C 229/08

(52) U.S. Cl. 560/155

(57) ABSTRACT

The invention relates to a method for producing optically active 3-aminocarboxylic acid ester compounds. According to said method, an enantiomer mixture of a mono-N-acylated 3-aminocarboxylic acid ester, which mixture was previously enriched in an enantiomer, is subjected to deacylation and then to a further enantiomer enrichment by crystallization by adding an acidic salt-forming substance.

The present invention relates to a method for preparing optically active 3-amino carboxylic ester compounds, and derivatives thereof.

Asymmetric synthesis, i.e. reactions in which a chiral group is generated from a prochiral one in such a way that the stereoisomeric products (enantiomers or diastereomers) result in unequal quantities, has become immensely important in particular in the pharmaceutical industry sector, because frequently only one particular optically active isomer has therapeutic activity. In this connection, optically active intermediates of active ingredients are also becoming increasingly important. This also applies to 3-amino carboxylic esters (formula I), and derivatives thereof, and especially also to 3-aminobutyric esters (formula II).

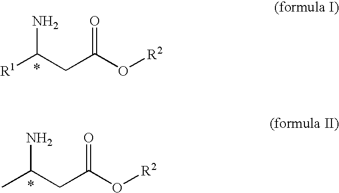

There is thus a great need for efficient synthetic routes for preparing optically active compounds of the general formulae I and II.

Several routes for preparing unsaturated 3-acetylamino carboxylic esters are described in the literature. It is known that enamines are obtained from β-keto esters by reaction with aqueous or gaseous ammonia. It is possible in a second step for an enamine prepared in this way to be N-acylated by reaction with acetic anhydride.

S. P. B. Ovenden et al. (J. Org. Chem. 1999, 64, 1140-1144) describe the one-stage preparation of α-unsaturated 3-acetylamino carboxylic esters by azeotropic dehydration of a solution, acidified with p-toluenesulfonic acid, of acetamide and of a β-keto ester in toluene or benzene.

The hydrogenation of olefins or β-substituted α-acylamidoacrylic acids is sufficiently well known to the skilled worker and is described for example in U.S. Pat. No. 3,849,480 and U.S. Pat. No. 4,261,919 respectively. W. S. Knowles and M. J. Sabacky disclose therein a general method for the homogeneously catalyzed, asymmetric hydrogenation of olefins (especially of β-substituted α-acylamidoacrylic acids) in the presence of an optically active hydrogenation catalyst, where an optically active enantiomeric form is required as product, and the metal of the catalyst complex is selected from Rh, Ir, Ru, Os, Pd and Pt.

Examples of the asymmetric hydrogenation of α-unsaturated 3-acetylamino carboxylic acid derivatives to saturated 3-amino carboxylic acid derivatives and of the chiral catalysts used for this purpose are disclosed inter alia in WO 9959721, WO 00118065, EP 967015, EP 1298136, WO 03031456 and WO 03042135.

N. W. Boaz et al. describe in Org. Proc. Res. Develop. 2005, 9, p. 472, the direct deacylation of 2-acetylamino carboxylic alkyl esters to 2-amino carboxylic alkyl esters. Reaction of the homologous 3-amino carboxylic alkyl esters is not described.

The present invention is therefore based on the object of providing a simple and thus economic method for preparing optically active 3-amino carboxylic esters and derivatives thereof.

It has now surprisingly been found that the stated object is achieved by a method in which a mono N-acylated 3-amino carboxylic ester is subjected to a deacylation and enantiomeric enrichment by crystallization.

The invention therefore relates to a method for preparing optically active 3-amino carboxylic ester compounds of the general formula I, and the ammonium salts thereof,

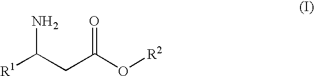

in which
R¹ is alkyl, cycloalkyl, heterocycloalkyl, aryl, or hetaryl, and
R² is alkyl, cycloalkyl or aryl,
in which a mixture of enantiomers, enriched in one enantiomer, of a mono N-acylated 3-amino carboxylic ester of the general formula (I.b),

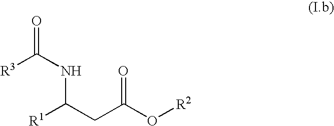

in which R¹ and R² have the meanings indicated above, and R³ is hydrogen, alkyl, cycloalkyl or aryl, is subjected to a deacylation by addition of an acidic salt former, and to a subsequent further enantiomeric enrichment by crystallization.

The present invention further relates to a method for preparing optically active 3-amino carboxylic ester compounds of the general formula I′, and derivatives thereof,

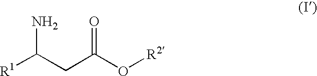

in which
R¹ is alkyl, cycloalkyl, heterocycloalkyl, aryl, or hetaryl, and
R^{2′} is hydrogen, a cation equivalent M⁺, alkyl, cycloalkyl or aryl, in which
   a) a β-keto ester of the general formula I.1

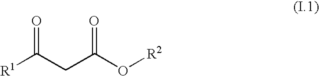

in which R¹ and R² have the meanings indicated above, is reacted
a 1) with at least one carboxamide of the formula R³—C(O)NH₂ in which R³ has the aforementioned meaning, in the presence of an amidation catalyst, or
a 2) with ammonia and subsequently with a carboxylic acid derivative of the formula R³—C(O)X in which X is halogen or a radical of the formula OC(O)R⁴ in which R⁴ has the meaning indicated above for R³,
to obtain the corresponding mixture of N-acylated, α-unsaturated (Z)- and (E)-3-amino carboxylic esters, and optionally the (Z)-3-amino carboxylic esters of the general formula (I.a) is isolated

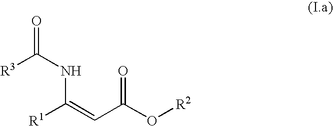

in which R¹, R² and R³ have the meanings indicated above,
b) the enamide (I.a) obtained in this reaction is subjected to an enantioselective hydrogenation in the presence of a chiral hydrogenation catalyst to obtain a mixture of enantiomers, enriched in one enantiomer, of mono N-acylated β-amino carboxylic esters of the general formula (I.b),

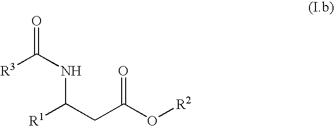

in which R¹, R² and R³ have the meanings indicated above,
c) the mixture of enantiomers, obtained in the hydrogenation, of the compounds I.b is subjected to a deacylation by adding an acidic salt former, and to a subsequent further enantiomeric enrichment by crystallization, and the ammonium salt of a 3-amino carboxylic ester which is formed in this way and is enriched in one stereoisomer is isolated, and
d) optionally the isolated ammonium salt is converted into the 3-amino carboxylic ester, and
e) optionally the 3-amino carboxylic ester is converted into the free 3-amino carboxylic acid or a salt thereof.

“Chiral compounds” are in the context of the present invention compounds having at least one chirality center (i.e. at least one asymmetric atom, for example at least one asymmetric C atom or P atom), with chirality axis, chirality plane or helical twist. The term “chiral catalyst” comprises catalysts which have at least one chiral ligand.

“Achiral compounds” are compounds which are not chiral.

A “prochiral compound” means a compound having at least one prochiral center. “Asymmetric synthesis” refers to a reaction in which a compound with at least one chirality center, one chirality axis, chirality plane or helical twist is generated from a compound with at least one prochiral center, with the stereoisomeric products resulting in unequal amounts.

“Stereoisomers” are compounds of identical constitution but different arrangement of atoms in three-dimensional space.

“Enantiomers” are stereoisomers which are related to one another as image to mirror image. The “enantiomeric excess” (ee) achieved in an asymmetric synthesis results from the following formula: ee[%]=(R−S)/(R+S)×100. R and S are the descriptors of the CIP system for the two enantiomers and represent the absolute configuration at the asymmetric atom. The enantiopure compound (ee=100%) is also referred to as “homochiral compound”.

The method of the invention leads to products which are enriched in a particular stereoisomer. The “enantiomeric excess” (ee) achieved is ordinarily at least 3% above that of the N-acylated 3-amino carboxylic ester. The value of ee which is achieved with the method is ordinarily at least 98%.

“Diastereomers” are stereoisomers which are not enantiomers of one another.

Although further asymmetric atoms may be present in the compounds encompassed by the present invention, the stereochemical terms detailed herein relate, unless expressly mentioned otherwise, to the carbon atom of the respective compounds which corresponds to the asymmetric β carbon atom in compound I or I′. If further stereocenters are present, in the context of the present invention they are ignored in the naming.

The term “alkyl” hereinafter comprises straight-chain and branched alkyl groups. These are preferably straight-chain or branched C₁-C₂₀-alkyl, more preferably C₁-C₁₂-alkyl, particularly preferably C₁-C₈-alkyl and very particularly preferably C₁-C₆-alkyl groups. Examples of alkyl groups are in particular methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-methylheptyl, nonyl, decyl, 2-propylheptyl.

The term “alkyl” also comprises substituted alkyl groups which may generally have 1, 2, 3, 4 or 5, preferably 1, 2 or 3 and particularly preferably 1, substituents selected from the groups cycloalkyl, aryl, hetaryl, halogen, COOR^{f}, COO⁻M⁺ and NE¹E², where R^{f} is hydrogen, alkyl, cycloalkyl or aryl, M⁺ is a cation equivalent, and E¹ and E² are independently of one another hydrogen, alkyl, cycloalkyl or aryl.

The term “cycloalkyl” comprises for the purposes of the present invention unsubstituted and substituted cycloalkyl groups, preferably C₃-C₈-cycloalkyl groups such as cyclopentyl, cyclohexyl or cycloheptyl, which in the event of substitution may generally have 1, 2, 3, 4 or 5, preferably 1, 2 or 3 and particularly preferably 1, substituents, preferably selected from among alkyl and the substituents mentioned for alkyl.

The term “heterocycloalkyl” for the purposes of the present invention comprises saturated cycloaliphatic groups having in general 4 to 7, preferably 5 or 6, ring atoms in which 1 or 2 of the ring carbon atoms are replaced by heteroatoms, preferably selected from the elements oxygen, nitrogen and sulfur, and which may optionally be substituted, where in the event of substitution these heterocycloaliphatic groups may have 1, 2 or 3, preferably 1 or 2, particularly preferably 1, substituents selected from alkyl, cycloalkyl, aryl, COOR^{f}, COO⁻M⁺ and NE¹E², preferably alkyl, where R^{f} is hydrogen, alkyl, cycloalkyl or aryl, M⁺ is a cation equivalent, and E¹ and E² are independently of one another hydrogen, alkyl, cycloalkyl or aryl. Examples which may be mentioned of such heterocycloaliphatic groups are pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, tetrahydrofuranyl, tetrahydropyranyl, dioxanyl.

The term “aryl” comprises for the purposes of the present invention unsubstituted and substituted aryl groups and stands preferably for phenyl, tolyl, xylyl, mesityl, naphthyl, fluorenyl, anthracenyl, phenanthrenyl or naphthacenyl, particularly preferably for phenyl or naphthyl, where these aryl groups may in the event of substitution have in general 1, 2, 3, 4 or 5, preferably 1, 2 or 3 and particularly preferably 1, substituents selected from the groups alkyl, alkoxy, nitro, cyano or halogen.

The term “hetaryl” comprises for the purposes of the present invention unsubstituted or substituted heterocycloaromatic groups, preferably the groups pyridyl, quinolinyl, acridinyl, pyridazinyl, pyrimidinyl, pyrazinyl, pyrrolyl, imidazolyl, pyrazolyl, indolyl, purinyl, indazolyl, benzotriazolyl, 1,2,3-triazolyl, 1,3,4-triazolyl and carbazolyl, where these heterocycloaromatic groups may in the event of substitution have in general 1, 2 or 3 substituents selected from the groups alkyl, alkoxy, acyl, carboxyl, carboxylate, —SO₃H, sulfonate, NE¹E², alkylene-NE¹E² or halogen, E¹ and E² being as defined above.

The above explanations of the terms “alkyl”, “cycloalkyl”, “aryl”, “heterocycloalkyl” and “hetaryl” apply correspondingly to the terms “alkoxy”, “cycloalkoxy”, “aryloxy”, “heterocycloalkoxy” and “hetaryloxy”.

The term “acyl” for the purposes of the present invention is alkanoyl or aroyl groups having in general 2 to 11, preferably 2 to 8, carbon atoms, for example the acetyl, propanoyl, butanoyl, pentanoyl, hexanoyl, heptanoyl, 2-ethylhexanoyl, 2-propylheptanoyl, benzoyl, naphthoyl or trifluoroacetyl group.

“Halogen” is fluorine, chlorine, bromine and iodine, preferably fluorine, chlorine and bromine.

M⁺ is a cation equivalent, i.e. a monovalent cation or the part of a polyvalent cation corresponding to a single positive charge. These include for example Li, Na, K, Ca and Mg.

The methods of the invention make it possible, as described above, to prepare optically active compounds of the general formula I and II, and to prepare derivatives thereof.

R¹ is preferably C₁-C₆-alkyl, C₃-C₇-cycloalkyl or C₆-C₁₄-aryl, each of which may optionally be substituted as stated at the outset. R¹ is in particular methyl, ethyl, n-propyl, isopropyl, n-butyl, tert-butyl, cyclohexyl or phenyl, specifically methyl.

R² is preferably unsubstituted or substituted C₁-C₆-alkyl, C₃-C₇-cycloalkyl or C₆-C₁₄-aryl. Particularly preferred radicals R² are methyl, ethyl, n-propyl, isopropyl, n-butyl, tert-butyl, trifluoromethyl, cyclohexyl, phenyl and benzyl.

R^{2′} is hydrogen, M⁺ and the meanings mentioned for R².

R³ is hydrogen, alkyl, cycloalkyl or aryl, in particular hydrogen, methyl, ethyl, trifluoromethyl, benzyl and phenyl.

According to the invention, a mixture of enantiomers of the compounds I.b is subjected to a deacylation by adding an acidic salt former and to a subsequent further enantiomeric enrichment by crystallization, and the ammonium salt of a 3-amino carboxylic ester which is formed in this way and is enriched in one stereoisomer is isolated.

It is a characteristic feature of the method of the invention that the isomer mixture of compounds of the general formula I.b employed for the deacylation also comprises the corresponding enantiomer or, starting from chiral β-keto esters, also diastereomers in non-negligible amounts. The method thus advantageously makes it possible to prepare optically active compounds of general formula I starting from isomer mixtures of compounds of the general formula I.b as are obtainable for example from the precursor compounds by conventional asymmetric hydrogenation of enamides.

The mixtures of enantiomers normally employed in this step of the method are already enriched in one enantiomer. The value of ee for these mixtures is preferably greater than 75% and particularly preferably greater than 90%.

In a preferred embodiment of the method of the invention, the deacylation is carried out in an alcoholic solvent.

An alcoholic solvent used according to the invention means both pure alcohols and solvent mixtures which comprise alcohols. These are in particular methanol, ethanol, n-propanol, isopropanol, n-butanol, t-butanol and cyclohexanol, and mixtures thereof with inert solvents such as aromatic compounds, for example toluene, and chlorinated hydrocarbons, dichloromethane or chloroform. A compound of the formula R²—OH is particularly preferred, with R² having the same meaning as in the product of the formula I or II.

In a further preferred embodiment of the method of the invention, at least one ester or a solvent mixture comprising at least one ester is added as solvent for the enantiomeric enrichment by crystallization. The ester preferably takes the form of alkyl acetates, in particular alkyl acetates of the formula CH₃C(O)OR² in which R² has the meaning given above. It is particularly preferred for R² to have the same meaning as in the reacted N-acylated 3-amino carboxylic ester of the formula (I.b). The ester is specifically methyl acetate or ethyl acetate.

In a specific embodiment of the invention, the solvent or solvent mixture used in the deacylation is, after the deacylation has taken place, partly or completely removed by a conventional method known to the skilled worker, specifically by a distillative method. Subsequently, for the enantiomeric enrichment by crystallization, a suitable solvent or solvent mixture, specifically consisting of or comprising an ester, is added to the residue. The solvent used for the enantiomeric enrichment by crystallization is preferably added to a concentrated (i.e. a saturated or almost saturated) solution of the 3-amino carboxylic ester compound. Subsequently, optionally, the residual content of the solvent used in the deacylation is further reduced by a method known to the skilled worker, preferably by distillation. It is particularly preferred in this connection for the residual content of the solvent used in the deacylation to be reduced to less than 5%.

The deacylation is preferably carried out at a temperature of at least 60° C., particularly preferably of at least 75° C. This temperature can be reduced for the subsequent crystallization.

The pressure in the deacylation is generally in a range from ambient pressure to 25 bar. When alcoholic solvents are employed, the pressure is preferably in a range from 1 to 10 bar. The subsequent crystallization can be carried out under atmospheric pressure.

In a preferred embodiment of the present invention, the salt former employed for the deacylation and for the subsequent crystallization is selected from achiral acidic compounds. Examples of suitable salt formers are acids which have a greater acidic strength than acidic acid in aqueous medium and form ammonium salts with the saturated β-amino carboxylic esters. The precipitation of the salts and their subsequent isolation advantageously leads to an increase in the optical purity.

The resulting salts of these salt formers are preferably selected from benzoate, oxalate, phosphate, sulfate, hydrogen oxalate, hydrogen sulfate, formate, lactate, fumarate, chloride, bromide, trifluoroacetate, p-toluenesulfonate and methanesulfonate. Suitable and particularly preferred are p-toluenesulfonate and methanesulfonate.

When such salt formers are employed, values of at least 98% for the ee are usually achieved for the isolated ammonium salt.

In a particularly preferred embodiment of the method of the invention, the salt former employed for the deacylation and for the subsequent crystallization is p-toluenesulfonate or methanesulfonate, and the alcoholic solvent used for the deacylation comprises a compound of the formula R²—OH where R² has the meaning given above.

The temperature during the enantiomeric enrichment by crystallization is generally in the range between the melting point and boiling point of the solvent or solvent mixture employed. In a suitable embodiment, the temperature can be increased and/or reduced one or more times during the crystallization in order to initiate crystal formation and/or to complete precipitation of the desired enantiomer.

The solid isolated after the enantiomer-enriching crystallization advantageously has a value of at least 97.0% and in particular of greater than 98% for the ee.

When N-acylated 3-amino carboxylic esters having a value of 95% for the ee are employed, the values of ee usually obtained for the corresponding ammonium salts after deacylation has taken place are at least 98%.

The product of the formula I or II obtained in the crystallization can be subjected to a working up (see the following statements about steps d) and e) of the method).

The invention further relates to a method comprising reaction stages a) to c) and optionally d) and e) described below.

### Stage a)

In one embodiment of stage a) of the method of the invention, a β-keto ester of the formula I.1 is reacted with at least one carboxamide of the formula R³—C(O)NH₂, in the presence of an amidation catalyst with removal of the water of reaction to give a 3-amino carboxylic ester of the formula I.a (step a.1).

The carboxamides of the formula R³—C(O)NH₂ in step a. 1 are preferably acetamide, propionamide, benzamide, formamide or trifluoroacetamide, in particular benzamide or acetamide.

Solvents suitable for step a.1 are those which form with water a low-boiling azeotrope from which the water of reaction can be removed by separation methods known to the skilled worker (such as, for example, azeotropic distillation). These are in particular aromatic compounds such as toluene, benzene, etc., ketones such as methyl isobutyl ketone or methyl ethyl ketone etc., and haloalkanes such as chloroform. Toluene is preferably employed.

Examples of suitable amidation catalysts are acids such as p-toluenesulfonic acid, methanesulfonic acid, sulfuric acid or the like. Preference is given to the use of p-toluenesulfonic acid.

The reaction in step a. 1 of the method preferably takes place at a temperature in the range from 20 to 110° C., particularly preferably 60 to 90° C. The temperature in this case is particularly preferably above the boiling point of the solvent used under standard conditions.

Step a.1 of the method is normally carried out under a pressure of from 0.01 to 1.5 bar, in particular 0.1 to 0.5 bar. The amino carboxylic ester obtained in step a.1 can optionally be subjected to a purification by conventional methods known to the skilled worker, e.g. by distillation.

In an alternative embodiment, α-keto ester of the formula I.1 is reacted with aqueous ammonia and then with a carboxylic acid derivative of the formula R³—C(O)X to give the N-acylated, β-unsaturated (Z)-3-amino carboxylic ester (I.a) in which X is halogen or a radical of the formula OC(O)R⁴ in which R⁴ has the meaning indicated above for R³ (step a.2).

The carboxylic acid derivative is preferably selected from carboxylic acid chlorides where X is chlorine, and R³ has the meaning given above, or carboxylic anhydrides where X is OC(O)R⁴ and R⁴ preferably has the same meaning as R³, with particularly preferred carboxylic acid derivatives being acetyl chloride, benzoyl chloride or acetic anhydride.

The acylation in step a.2 is preferably carried out at a temperature in the range from 20° C. to 120° C., particularly preferably at a temperature in the range from 60° C. to 90° C.

The acylation in step a.2 is carried out in a polar solvent or a mixture of a polar solvent with a nonpolar solvent; the polar solvent is preferably a carboxylic acid of the formula R³COOH or a tertiary amine, and haloalkanes and aromatic compounds are particularly suitable as nonpolar solvents, with particular preference for use of acetic acid or triethylamine as solvent.

The acylation in step a.2 can be carried out with use of a catalyst which can be employed both in catalytic amounts and stoichiometrically or as solvent, with preference for non-nucleophilic bases such as tertiary amines, and with particular preference being given in this connection to triethylamine and/or dimethylaminopyridine (DMAP).

Steps a.1 and a.2 result optionally in the (Z)-3-amino carboxylic ester as mixture with the (E)-3-amino carboxylic ester and, optionally, further acylation products. In this case, the (Z)-3-amino carboxylic ester of the formula I.a will be isolated by processes known to the skilled worker. A preferred method is separation by distillation.

### Stage b)

The α-unsaturated (Z)-3-amino carboxylic ester compounds of the formula I.a which are obtained in stage a can subsequently be subjected to an enantioselective hydrogenation in the presence of a chiral hydrogenation catalyst to obtain a mixture of enantiomers, enriched in one enantiomer, of mono N-acylated β-amino carboxylic esters of the general formula (I.b).

The hydrogenation catalyst preferably employed in stage b) is at least one complex of a transition metal of groups 8 to 11 of the Periodic Table of the Elements, which comprises as ligand at least one chiral, phosphorus atom-containing compound.

The chiral hydrogenation catalyst employed for the hydrogenation is preferably able to hydrogenate the α-unsaturated, N-acylated 3-amino carboxylic ester (I.a) employed with preference for the desired isomer. The compound of the formula I.b obtained after the asymmetric hydrogenation in step b) preferably has a value for ee of at least 75%, particularly preferably at least 90%. However, such a high enantiomeric purity is in many cases unnecessary in the method of the invention, because a further enantiomeric enrichment takes place in the subsequent deacylation and crystallization step in the method of the invention. However, the value for ee of the compound I.b is preferably at least 75%.

The method of the invention preferably makes the enantioselective hydrogenation possible with substrate/catalyst ratios (s/c) of at least 1000:1, particularly preferably at least 5000:1 and especially at least 15000:1.

A complex of a metal of group 8, 9 or 10 with at least one of the ligands mentioned below is preferably employed for the asymmetric hydrogenation. The transition metal is preferably selected from Ru, Rh, Ir, Pd or Pt. Catalysts based on Rh and Ru are particularly preferred. Rh catalysts are especially preferred.

The phosphorus-containing compound employed as ligand is preferably selected from bidentate and multidentate phosphine, phosphinite, phosphonite, phosphoramidite and phosphite compounds.

Catalysts for the hydrogenation will preferably have at least one ligand which is selected from compounds of the following formulae

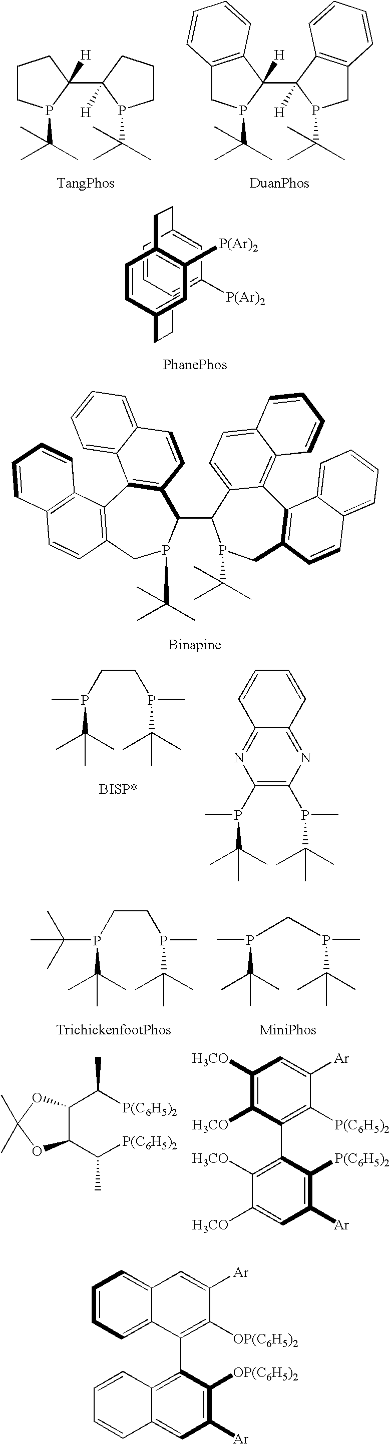

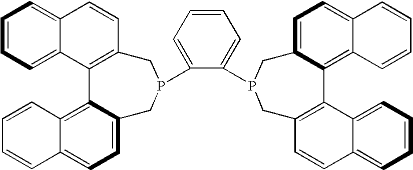

or their enantiomers, where Ar is optionally substituted phenyl, preferably tolyl or xylyl.

Bidentate compounds of the aforementioned compound classes are particularly preferred. P-chiral compounds such as DuanPhos, TangPhos or Binapine are especially preferred.

Suitable chiral ligands coordinating via at least one phosphorus atom to the transition metal are known to the skilled worker and are commercially available for example from Chiral Quest (Princeton) Inc., Monmouth Junction, N.J.). The naming of the chiral ligands listed by way of example above corresponds to their commercial designation.

Chiral transition metal complexes can be obtained in a manner known to the skilled worker (e.g. Uson, Inorg. Chim. Acta 73, 275 1983, EP-A-0 158 875, EP-A-437 690) by reacting suitable ligands with complexes of the metals which comprise labile or hemilabile ligands. The precatalysts which can be used in this connection are complexes such as, for instance, Pd₂(dibenzylideneacetone)₃, Pd(OAc)₂ (Ac=acetyl), RhCl₃, Rh(OAc)₃, [Rh(COD)Cl]₂, [Rh(COD)OH]₂, [Rh(COD)OMe]₂ (Me=methyl), Rh(COD)acac, Rh₄(CO)₁₂, Rh₆(CO)₁₆, [Rh(COD)₂)]X, Rh(acac)(CO)₂ (acac=acetylacetonato), RuCl₃, Ru(acac)₃, RuCl₂(COD), Ru(COD)(methallyl)₂, Ru(Ar)I₂ and Ru(Ar)Cl₂, Ar=aryl, both unsubstituted and substituted, [Ir(COD)Cl]₂, [Ir(COD)₂]X, Ni(allyl)X. It is also possible to use NBD (=norbornadiene) instead of COD (=1,5-cyclooctadiene). Preference is given to [Rh(COD)Cl]₂, [Rh(COD)₂)]X, Rh(acac)(CO)₂, RuCl₂(COD), Ru(COD)(methallyl)₂, Ru(Ar)Cl₂, Ar=aryl, both unsubstituted and substituted, and the corresponding systems with NBD instead of COD. [Rh(COD)₂)]X and [Rh(NBD)₂)]X are particularly preferred.

X can be any anion which is known to the skilled worker and can generally be used in asymmetric synthesis. Examples of X are halogens such as Cl⁻, Br⁻⁻ or I⁻, BF₄⁻, ClO₄⁻, SbF₆⁻, PF₆⁻, CF₃SO₃⁻, BAr₄⁻. X is preferably BF₄⁻, PF₆⁻, CF₃SO₃⁻, SbF₆⁻.

The chiral transition metal complexes can either be generated in situ in the reaction vessel before the actual hydrogenation reaction, or else be generated separately, isolated and subsequently employed. It is possible in this connection for at least one solvent molecule to undergo addition onto the transition metal complex. Common solvents (e.g. methanol, diethyl ether, tetrahydrofuran (THF), dichloromethane, etc.) for preparing the complexes are known to the skilled worker.

Phosphine-, phosphinite-, phosphonite-, phosphoramidite- and phosphite-metal or -metal-solvent complexes still having at least one labile or hemilabile ligand are suitable precatalysts from which the actual catalyst is generated under the conditions of the hydrogenation.

The hydrogenation step (step b) of the method of the invention is usually carried out at a temperature of from −10 to 150° C., preferably at 0 to 120° C. and particularly preferably at 10 to 70° C.

The hydrogen pressure can in this case be varied in a range between 0.1 bar and 600 bar. It is preferably in a pressure range from 0.5 to 20 bar, particularly preferably between 1 to 10 bar.

Solvents suitable for the hydrogenation reaction of the enamides from I.a are all solvents known to the skilled worker for asymmetric hydrogenation. Preferred solvents are lower alkyl alcohols such as methanol, ethanol, isopropanol, and toluene, THF, ethyl acetate. The solvent particularly preferably employed in the method of the invention is ethyl acetate or THF.

The hydrogenation catalysts (and precatalysts) described above can also be immobilized in a suitable manner, e.g. by attachment via functional groups suitable as anchor groups, by adsorption, grafting, etc., on a suitable support, e.g. composed of glass, silica gel, synthetic resins, polymeric supports, etc. They are then also suitable for use as solid-phase catalysts. It is advantageously possible by these methods to reduce the catalyst consumption further. The catalysts described above are also suitable for a continuous reaction process, e.g. after immobilization as described above, in the form of solid-phase catalysts.

In a further embodiment, the hydrogenation in stage b is carried out continuously. Continuous hydrogenation can take place in one or, preferably, in a plurality of reaction zones. A plurality of reaction zones can be formed by a plurality of reactors or by spatially different regions within one reactor. If a plurality of reactors is employed, the reactors may in each case be identical or different. They may in each case have identical or different mixing characteristics and/or be subdivided one or more times by internals. The reactors can be connected together as desired, e.g. in parallel or in series.

Suitable pressure-resistant reactors for hydrogenation are known to the skilled worker. These include the generally customary reactors for gas-liquid reactions, such as, for example, tube reactors, tube bundle reactors, stirred vessels, gas circulation reactors, bubble columns, etc., which may optionally be packed or subdivided by internals.

### Step c)

Concerning step c), reference is made to the statements made at the outset about the crystallization by adding an acidic salt former.

### Step d)

If desired, the ammonium salts isolated in the enantiomer-enriching crystallization can be subjected to a further working up. Thus, it is possible to liberate the optically active compound of the formula I by contacting the product of the crystallization with a suitable base, preferably NaHCO₃, NaOH, KOH. In a suitable procedure, the product of the crystallization is dissolved or suspended in water and then the pH is adjusted to about 8 to 12, preferably about 10, by addition of bases. The free 3-amino carboxylic ester can be isolated by extracting the basic solution or suspension with a suitable organic solvent, e.g. an ether such as methyl butyl ether, a hydrocarbon or hydrocarbon mixture, e.g. an alkane, such as pentane, hexane, heptane, or an alkane mixture, ligroin or petroleum ether, or aromatic compounds such as toluene. A preferred extractant is toluene. It is possible with this procedure to obtain the 3-amino ester virtually quantitatively, with the value of the ee also being retained.

### Step e)

It is possible optionally for the 3-amino carboxylic esters to be derivatized using methods known to the skilled worker. Possible derivatizations comprise for example hydrolysis of the ester or stereoselective reduction of the carboxyl carbon atom to an optically active alcohol.

Derivatives according to the invention of compounds of the formula I′ thus comprise for example ammonium salts of the 3-amino carboxylic esters, the free carboxylic acid in which R^{2′} is hydrogen, salts of the free carboxylic acid in which R^{2′} is M⁺, and optically active 3-amino alcohols.

In a specific embodiment, the method described above is used to prepare optically active compounds of the formula II, or the ammonium salts thereof, with the following absolute configuration, or to prepare the enantiomers of these compounds or salts,

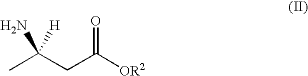

where R² is C₁-C₆-alkyl. These compounds and their salts are obtained in high optical purity, in particular with a value of at least 98% for the ee.

### EXAMPLES

### Example 1

### Preparation of methyl (R)-3-aminobutyrate

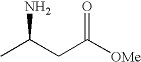

### a) Synthesis of methyl (Z)-N-acetyl-3-aminocrotonate

A solution of methyl acetoacetate (580 g, 5 mol), acetamide (295 g, 5 mol) and p-toluenesulfonic acid monohydrate (19 g, 0.1 mol) in toluene (1 l) was heated at a reflux temperature of 80° C. and a pressure of 300 mbar with a water trap until water of reaction no longer separated out and GC analysis confirmed that reaction was complete (24 h). After the reaction mixture was cooled to 25° C., the organic phase was washed with water (2×375 ml). The combined aqueous phases were then extracted with toluene (500 ml), the collected organic phases were combined, and the toluene was removed under reduced pressure. The crude product obtained in this way was purified by distillation under reduced pressure (15 mbar) through a short column with a distillate temperature of 105° C. Methyl (Z)-N-acetyl-3-aminocrotonate (380 g, 2.38 mmol) was obtained in a purity of 98% (GC). The yield was 47%.

### b) Synthesis of methyl (R)—N-acetyl-3-aminobutyrate

Under protective gas, methyl (Z)-N-acetyl-3-aminocrotonate (200 g, 1.27 mmol) was dissolved in THF (200 g) and degassed by brief evacuation of the reaction vessel. After addition of [Rh(COD)DuanPhos]OTf (31.5 mg, 0.042 mmol), the resulting solution was transferred while continuing to maintain the protective gas atmosphere into a 1.2 l autoclave. The autoclave was flushed twice with a hydrogen pressure of 5 bar and then heated under this hydrogen pressure to 70° C. and stirred for 20 h. GC analysis of the discharge from the reaction showed a conversion of 99.5% with a methyl (R)—N-acetyl-3-aminobutyrate content of 99.2%. The value of ee was 95.1%.

### c) Synthesis of methyl (R)-3-aminobutyrate x p-toluenesulfonic acid

The solvent was removed from a solution of methyl (R)—N-acetyl-3-aminobutyrate (37.5 g) in THF (43 ml) obtained in stage b) at a temperature of 50° C. under reduced pressure. The residue was taken up in methanol (94 ml) and, after addition of p-toluenesulfonic acid monohydrate (53.8 g), stirred under autogenous pressure at 100° C. for 12 h. Cooling of the reaction solution and decompression was followed by removal of the methanol under reduced pressure at 50° C. The residue was mixed with methyl acetate (112 ml) at 50° C. and then slowly cooled to 0-5° C. The precipitated product was isolated by filtration, washed with cold methyl acetate and then dried in vacuo.

The structure of the compound was verified by NMR spectroscopy. The content of the compound was determined by titration with a base. The enantiomeric purity was determined after derivatization by gas chromatography on a chiral phase. The derivatization of amino acids and their derivatives for determining the enantiomeric purity is known to the skilled worker.

The reaction products were analyzed by GC using the following method:

### Determination of Conversion:

Separating column: 25 m*0.32 mm OV 1, FD=0.5 μm, 50°, 2′, 20°/′, 300°, 45°.
Starting material: 8.1 min; product: 8.3 min

### Determination of ee:

Precolumn: 25 m*0.25 mm Optima-1, FD=0.5 μm; chir. column: 30 m*25 mm BGB 174S;
FD=0.25 um; Temp. program: 140° C., 12′; 10° C./min, 200° C., 2 min; Col 1:
Ramp.press.: 1.7 bar H2 (4.6 ml/′); 1.7 min, 10 bar/min, 1.9 bar, 0.2 min; 10 bar/min; 1.4 bar; Col 2: Const. press., 1.3 bar H2 (2.7 ml/min).

### Methyl (R)-3-N-acetylaminobutyrate: 9.87 min

Methyl (S)-3-N-acetylaminobutyrate: 10.51 min

1. A method for preparing optically active 3-amino carboxylic ester compounds of the general formula I, and the ammonium salts thereof, 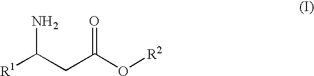
in which
R¹ is alkyl, cycloalkyl, heterocycloalkyl, aryl, or hetaryl, and
R² is alkyl, cycloalkyl or aryl,
in which a mixture of enantiomers, enriched in one enantiomer, of a mono N-acylated 3-amino carboxylic ester of the general formula (I.b), 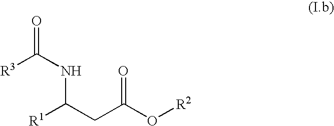
in which R¹ and R² have the meanings indicated above, and R³ is hydrogen, alkyl, cycloalkyl or aryl, is subjected to a deacylation by addition of an acidic salt former, and to a subsequent further enantiomeric enrichment by crystallization.

2. A method for preparing optically active 3-amino carboxylic ester compounds of the general formula I′, and derivatives thereof, 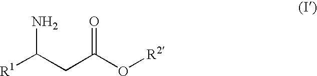
in which
R¹ is alkyl, cycloalkyl, heterocycloalkyl, aryl, or hetaryl, and
R^{2′} is hydrogen, a cation equivalent M⁺, alkyl, cycloalkyl or aryl, in which
a) a β-keto ester of the general formula I.1 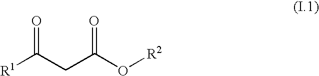
in which R¹ and R² have the meanings indicated above, is reacted
a 1) with at least one carboxamide of the formula R³—C(O)NH₂ in which R³ is hydrogen, alkyl, cycloalkyl, or aryl, in the presence of an amidation catalyst, or
a 2) with ammonia and subsequently with a carboxylic acid derivative of the formula R³—C(O)X in which X is halogen or a radical of the formula OC(O)R⁴ in which R⁴ has the meaning indicated above for R³,
to obtain the corresponding N-acylated, α-unsaturated (Z)-3-amino carboxylic esters of the general formula (I.a) 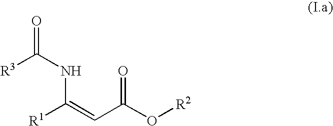
in which R¹, R² and R³ have the meanings indicated above,
b) the enamide (I.a) obtained in this reaction is subjected to an enantioselective hydrogenation in the presence of a chiral hydrogenation catalyst to obtain a mixture of enantiomers, enriched in one enantiomer, of mono N-acylated β-amino carboxylic esters of the general formula (I.b), 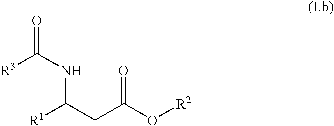
in which R¹, R² and R³ have the meanings indicated above,
c) the mixture of enantiomers, obtained in the hydrogenation, of the compounds I.b is subjected to a deacylation by adding an acidic salt former, and to a subsequent further enantiomeric enrichment by crystallization, and the ammonium salt of a 3-amino carboxylic ester which is formed in this way and is enriched in one stereoisomer is isolated, and
d) optionally the isolated ammonium salt is converted into the 3-amino carboxylic ester, and
e) optionally the 3-amino carboxylic ester is converted into the free 3-amino carboxylic acid or a salt thereof.

3. The method according to claim 2, where a β-keto ester of the formula I.1 is reacted with at least one carboxamide of the formula R³—C(O)NH₂ in the presence of an amidation catalyst with removal of the water of reaction to give a 3-amino carboxylic ester of the formula I.a.

4. The method according to claim 1, where the deacylation is carried out in an alcoholic solvent.

5. The method according to claim 1, where the enantiomer-enriching crystallization is carried out with addition of an ester.

6. The method according to claim 1, where the salt former employed for the deacylation and for the crystallization is selected from achiral acidic compounds.

7. The method according to claim 6, where the salt former is selected from p-toluenesulfonic acid, methanesulfonic acid, benzoic acid, oxalic acid, phosphoric acid, sulfuric acid, hydrogen oxalate, hydrogen sulfate, formic acid, lactic acid, fumaric acid, hydrochloric acid, hydrobromic acid and trifluoroacetic acid.

8. A method according to claim 1, where the salt former employed for the deacylation and for the crystallization is p-toluenesulfonic acid or methanesulfonic acid, and the alcoholic solvent used for the deacylation comprises a compound of the formula R²—OH where R² is alkyl cycloalkyl or aryl.

9. The method according to claim 2, where the hydrogenation catalyst employed is at least one complex of a transition metal of groups 8 to 11 of the Periodic Table of the Elements which comprises as ligand at least one chiral phosphorus atom-containing compound.

10. The method according to claim 9, where the transition metal is selected from Ru, Rh, Ir, Pd or Pt.

11. The method according to claim 9, where the catalyst has at least one ligand which is selected from bidentate and multidentate phosphine, phosphinite, phosphonite, phosphoramidite and phosphite compounds.

12. The method according to claim 11, where the catalyst has at least one ligand which is selected from compounds of the following formulae 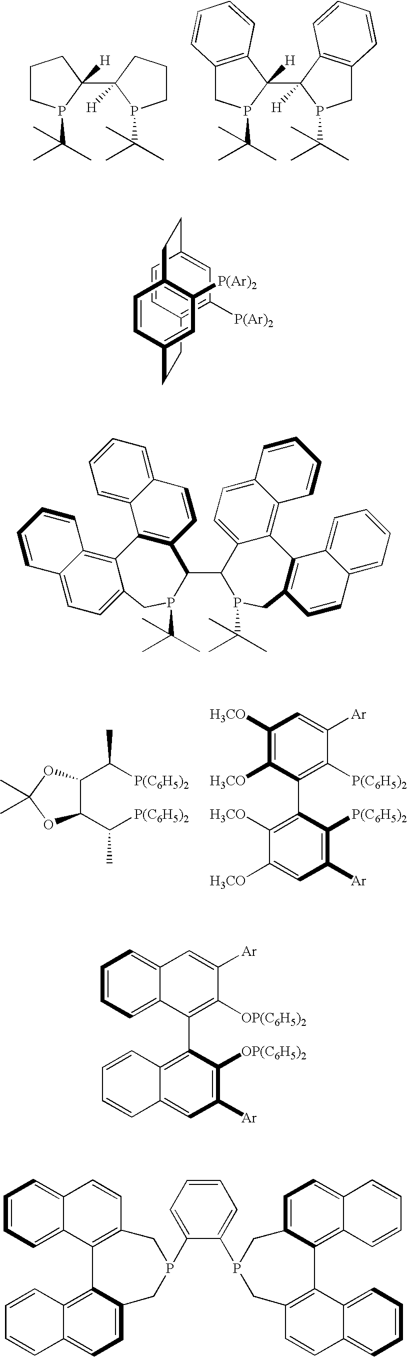
or their enantiomers, where Ar is tolyl or xylyl.

13. The method according to claim 2, where at least one of the steps of the method is carried out continuously.

14. The method according to claim 13, where the hydrogenation is carried out continuously.

15. The method according to claim 1, where R is C₁-C₉-alkyl, and R² and R³ have the meanings mentioned in claim 1.

16. The method according to claim 1, where R³ is methyl, and R¹ and R² have the meanings mentioned in claim 1.

17. The method according to claim 1 where the value of ee for the solid isolated after the crystallization is at least 98%.

18. The method according to claim 1, where an optically active compound of the formula II or the ammonium salts thereof with the following absolute configuration, or the optically active enantiomer of this compound or salts, is obtained, 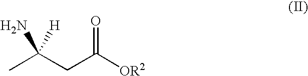
where R² is C₁-C₆-alkyl.
